(19) United States
(12) Patent Application Publication
(10) Pub. No.: US 2014/0124668 A1
(43) Pub. Date: May 8, 2014

(54) RADIATION IMAGING APPARATUS, OPERATION ASSISTING APPARATUS, CONTROL METHODS THEREOF, AND STORAGE MEDIUM

(71) Applicant: CANON KABUSHIKI KAISHA, Tokyo (JP)

(72) Inventors: Taisuke Ikawa, Yokohama-shi (JP); Kazumasa Matsumoto, Yokohama-shi (JP)

(73) Assignee: CANON KABUSHIKI KAISHA, Tokyo (JP)

(21) Appl. No.: 14/068,142

(22) Filed: Oct. 31, 2013

(30) Foreign Application Priority Data
Nov. 8, 2012 (JP) 2012-246662

(51) Int. Cl. G01T 1/16; A61B 6/12

(52) U.S. Cl. CPC G01T 1/16; A61B 6/12
USPC 250/336.1; 600/424

(57) ABSTRACT

A radiation imaging apparatus that captures a radiation image of an object, comprising: an output unit configured to, in accordance with a driving status of the radiation imaging apparatus, output a signal to restrict driving of an operation assisting apparatus that generates a magnetic field when assisting an operation for the object.

### BACKGROUND OF THE INVENTION

1. Field of the Invention

The present invention relates to a radiation imaging apparatus, an operation assisting apparatus, control methods thereof, and a storage medium.

2. Description of the Related Art

Recently, a technique called IVR (Interventional radiology), in which a surgical operation is carried out while confirming diagnostic images, is becoming widespread in the medical field. Accordingly, a method of diagnosing/treating a region of interest by guiding a catheter up to a region of interest while observing/diagnosing radiation images of an object has become popular.

In the above-described method, however, radiation imaging needs to be frequently done during the operation, and the radiation dose increases. In U.S. Pat. No. 6,233,476, a method of detecting the relative position of a catheter by magnetic targeting and plotting the end position of the catheter on a radiation image captured in advance is used. Since this method can detect the catheter position even in a non-exposure state, the radiation dose can be suppressed.

In such an operation system, however, the radiation imaging apparatus and the operation assisting apparatus that detects the relative position of the catheter by the operator's manipulation are separately driven. Hence, the object may be captured in a state in which the operation assisting apparatus is being driven. Since the operation assisting apparatus generates a magnetic field having a high frequency and a high intensity, the acquired radiation image may include noise (for example, stripe-shaped noise). The higher the frequency the power supplied to the operation assisting apparatus has, the more conspicuous its influence is.

### SUMMARY OF THE INVENTION

According to one aspect of the present invention, there is provided a radiation imaging apparatus that captures a radiation image of an object, comprising:

an output unit configured to, in accordance with a driving status of the radiation imaging apparatus, output a signal to restrict driving of an operation assisting apparatus that generates a magnetic field when assisting an operation for the object.

Further features of the present invention will be apparent from the following description of exemplary embodiments with reference to the attached drawings.

### BRIEF DESCRIPTION OF THE DRAWINGS

FIG. 1 is a block diagram showing the schematic arrangement of a radiation imaging system according to the first embodiment;

FIG. 2 is a block diagram showing the internal arrangement of a magnetic field generator/detector 201 included in an operation assisting apparatus 20 according to the first embodiment;

FIG. 3 is a flowchart showing the procedure of processing executed by a radiation imaging apparatus 10 according to the first embodiment;

FIG. 4 is a table that defines whether to output a restriction signal in accordance with a restriction level and the driving status of the radiation imaging apparatus 10 according to the first embodiment;

FIG. 5 is a view showing an example of a restriction signal generation timing for each restriction level according to the first embodiment;

FIG. 6 is a perspective view showing an example of the installation positions of radiation detector magnetic sensors according to the second embodiment;

FIG. 7 is a block diagram showing the schematic arrangement of a radiation imaging system according to the third embodiment; and

FIG. 8 is a view showing an example of the arrangement of the radiation detector 102 according to the first embodiment.

### DESCRIPTION OF THE EMBODIMENTS

Exemplary embodiments of the present invention will now be described in detail with reference to the drawings. It should be noted that the relative arrangement of the components, the numerical expressions and numerical values set forth in these embodiments do not limit the scope of the present invention unless it is specifically stated otherwise.

The embodiments of the present invention will now be described with reference to the accompanying drawings.

### First Embodiment

FIG. 1 is a block diagram showing the schematic arrangement of a radiation imaging system according to the first embodiment. The radiation imaging system according to the first embodiment includes a radiation imaging apparatus 10 and an operation assisting apparatus 20.

<Arrangement of Radiation Imaging Apparatus 10>

The radiation imaging apparatus 10 includes a radiation tube 101, a radiation detector 102, a driving control unit 103, an image processing unit 104, a monitor 105, a synchronization control unit 106, and a high voltage generator 107.

The radiation tube 101 irradiates an object M with a radiation (for example, X-rays). Note that the radiation is not limited to X-rays, and the present invention is also applicable to another radiation such as α-rays, β-rays, or γ-rays. The radiation detector 102 detects the radiation transmitted through the object M. The radiation tube 101 and the radiation detector 102 are arranged to face each other. For example, the radiation tube 101 and the radiation detector 102 may be fixed by an arm so as to be rotatable about the object M as the arm rotates. The detection surface of the radiation detector 102 is provided with a number of detection elements (not shown) for detecting the radiation. The radiation detector 102 detects the radiation and generates a detection signal (charge information) for each detection element.

The driving control unit 103 controls read driving from each detection element of the radiation detector 102, and the radiation detector 102 thus collects the detection signals. The collected detection signals are output from the radiation detector 102 to the image processing unit 104 via an A/D converter (not shown).

The image processing unit 104 performs various kinds of processing such as correction for the detection signals, thereby acquiring image data. The acquired image data is stored in a storage unit (not shown) provided in the image processing unit 104 and also output to the monitor 105. The image processing unit 104 also calculates the noise amount of the image and decides the generation timing of a restriction signal (to be described later) to restrict driving of the operation assisting apparatus 20 based on the image noise amount. Note that the image processing unit 104 is implemented by a central processing unit (CPU) for executing various kind of processing and operations, and storage media such as a RAM (Random-Access Memory) serving as the work area of arithmetic processing and a fixed disk for storing various kinds of information. The monitor 105 displays a radiation image based on the acquired image data.

The synchronization control unit 106 is connected to the high voltage generator 107 and the radiation detector 102 directly or via the image processing unit 104, and synchronizes radiation irradiation with radiation detection. More specifically, the synchronization control unit 106 causes the high voltage generator 107 to apply a tube voltage to the radiation tube 101 in accordance with the timing of detection signal collection of the radiation detector 102. This makes it possible to indirectly give the radiation tube 101 an instruction to irradiate the object with the radiation at a predetermined irradiation time. The synchronization control unit 106 also controls the image processing unit 104 to synchronize image data output and the like with the radiation irradiation and radiation detection. The high voltage generator 107 applies a desired tube voltage to the connected radiation tube 101.

<Arrangement of Radiation Detector 102>

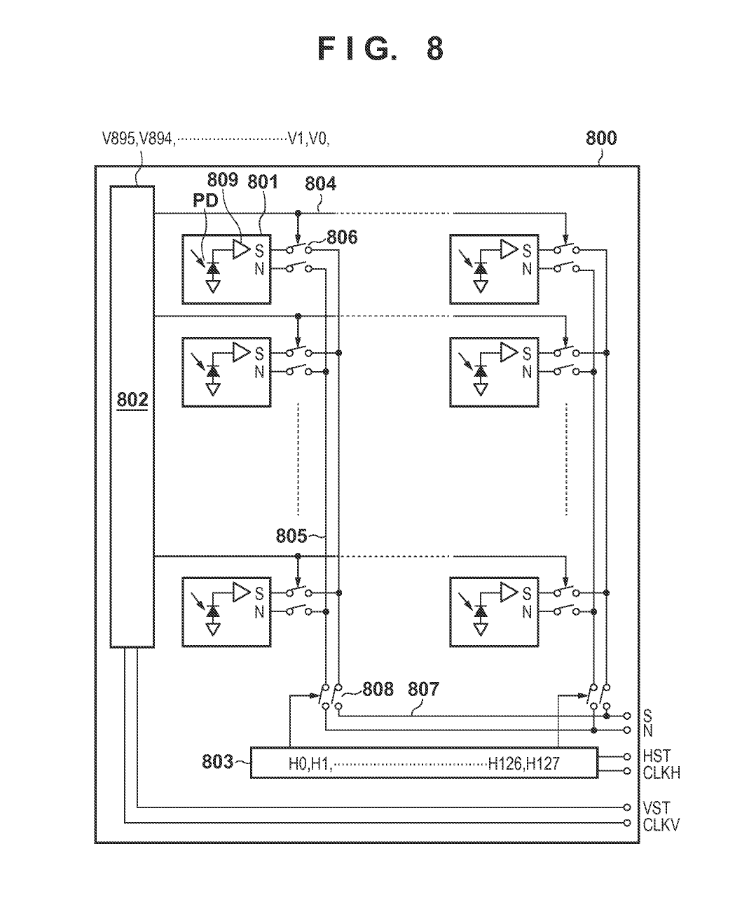

The detailed arrangement of the radiation detector 102 will be described with reference to FIG. 8. The radiation detector 102 includes a scintillator, including CsI:Tl, that converts a radiation into an electromagnetic wave of a different wavelength, for example, visible light, and a sensor 800 that is formed on a substrate of a-Si or single-crystal Si and converts visible light into an electrical signal. Note that the sensor 800 itself may have sensitivity using a-Se so as to detect a radiation and convert it into an electrical signal. In this case, the scintillator may be omitted.

The sensor 800 includes a plurality of pixels 801 arranged in a matrix and including detection elements, and a vertical shift register 802 and a horizontal shift register 803 each serving as a driving circuit. Each pixel 801 is connected, via a first switch 806, to a column signal line 805 that is shared by the pixels in the column direction. Each first switch 806 is on/off-controlled by a voltage applied to a corresponding row selection line 804. The timing of voltage application to each row selection line 804 is controlled by the vertical shift register 802.

There are provided a plurality of column signal lines 805 which are connected to a common analog output line 807 via second switches 808. The analog output line 807 is connected to an external terminal. The electrical signal from each pixel 801 is output externally from the senor via the analog output line 807. In this case, the output signal is an analog image signal. These signals are amplified by an amplifier and converted into digital data by an A/D converter. Radiation image data is thus obtained. Each second switch 808 is on/off-controlled by the horizontal shift register 803.

In accordance with a vertical shift register start VST input to the vertical shift register 802, the vertical shift register 802 controls the timing of applying, to the first row selection line 804, a voltage (on voltage) to turn on the first switch 806. In addition, the timing of applying the on voltage to the next row selection line 804 is controlled by a vertical shift clock signal CLKV. In accordance with a horizontal shift register start HST input to the horizontal shift register 803, the horizontal shift register 803 controls the timing of applying a voltage (on voltage) to turn on the first second switch 808. In addition, the timing of applying the on voltage to the next second switch 808 is controlled by a horizontal shift clock signal CLKH. The signals VST, CLKV, HST, and CLKH are output from, for example, the driving control unit 103. The driving control unit 103 thus controls the driving of the sensor.

The pixel 801 includes, for example, a detection element PD, and a pixel amplifier 809 that amplifies an electrical signal obtained by the detection element PD. The pixel amplifier 809 is, for example, a source follower. Transistors included in the source follower form an FD (Floating Diffusion) portion.

A sample hold circuit that holds the output of the pixel amplifier 809 may also be included. Two sample hold circuits can be provided to hold two, S and N signals. In this case, the column signal lines 805, the first switches 806, the analog output lines 807, and the second switches 808 are needed for the two, S and N systems. Additionally, in this case, a differential amplifier is used as the amplifier connected to the output terminal of the sensor. The difference signal between S and N can be obtained by the differential amplifier.

In addition, a reset circuit connected to the detection element of the pixel 801 may be provided. The reset circuit includes a transistor having one terminal connected to the detection element, the other terminal connected to GND, and the base connected to a control line.

The pixel amplifier 809, the sample hold circuit, and the reset circuit are controlled by, for example, the driving control unit 103.

At the time of image capturing, reset driving, an accumulation state, sampling driving, and readout driving are performed in this order. Reset driving is to reset the electrical signal accumulated in the parasitic capacitance or FD portion of the detection element under the control of the driving control unit 103. The accumulation state is a state in which the transistor of the reset circuit is set off, and charges are accumulated in the parasitic capacitance or FD portion of the detection element. The synchronization control unit 106 synchronizes the radiation detector 102 and the radiation tube 101 so as to perform radiation irradiation during this accumulation period. Sampling driving is driving that causes the sample hold circuit to sample and hold an electrical signal obtained by causing the pixel amplifier 809 to amplify the charges accumulated in the accumulation state. Readout driving indicates a series of driving operations in which the sampled and held electrical signal is output from the pixel 801 via the column signal line 805 and the analog output line 807, and the amplifier and the A/D converter outside the sensor are operated to obtain radiation image data.

Note that in the above-described arrangement, reset driving and sampling driving or reset driving and readout driving can be executed concurrently for a certain pixel. Similarly, the accumulation state and readout driving can be executed concurrently for a certain pixel. In this case, restriction signal output starts in accordance with the end of the accumulation period, that is, in accordance with the end of the X-ray irradiation period and the start of sampling driving. Note that the restriction signal output is done by, for example, the driving control unit 103.

Alternatively, electronic shutter driving may be executed in which reset driving is performed for all pixels at once, and sampling driving is also performed at once to make the accumulation periods of all pixels approximately match. Otherwise, rolling shutter driving may be executed in which reset and sampling are sequentially performed on the line basis. Note that the electronic shutter driving can shorten the period in which the driving of the operation assisting apparatus is restricted because reset driving or sampling driving is performed at almost the same timing in all pixels.

As described above, the radiation imaging apparatus includes the sensor that outputs an image signal by a radiation, and the driving control unit that controls the sensor. The driving control unit can cause the sensor to perform electronic shutter driving.

In another example, the pixel 801 does not include the internal amplifier and the sample hold circuit, and the detection element PD is directly connected to the first switch 806. In this case, reset driving is the same as readout driving as, for example, driving in the sensor 800. In this case, the amplifier and the A/D converter outside the sensor 800 are not operated. Sampling driving is not performed or is performed as one process outside the sensor 800 in readout driving.

<Arrangement of Operation Assisting Apparatus 20>

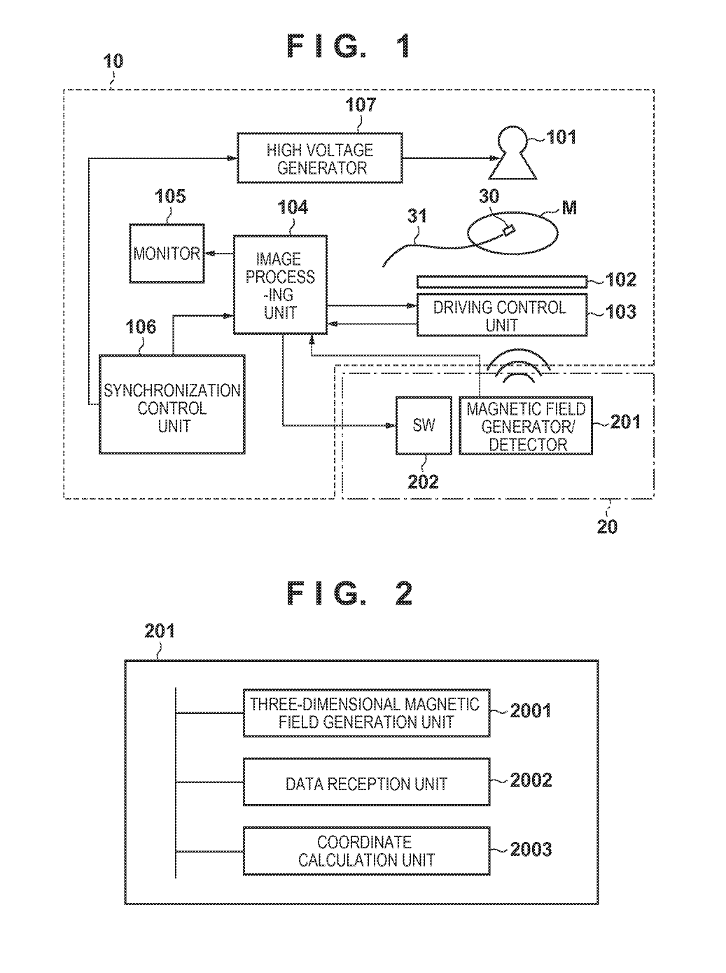

The arrangement of the operation assisting apparatus 20 according to the first embodiment will be described next with reference to FIGS. 1 and 2. The operation assisting apparatus 20 includes a magnetic field generator/detector 201 and a switch SW 202. As shown in FIG. 2, the magnetic field generator/detector 201 includes a three-dimensional magnetic field generation unit 2001, a data reception unit 2002, and a coordinate calculation unit 2003, and can detect the position of an operation assisting apparatus magnetic sensor 30. The operation assisting apparatus 20 is, for example, a catheter navigation system.

The three-dimensional magnetic field generation unit 2001 generates a magnetic field having amplitudes in three-dimensional directions. The operation assisting apparatus magnetic sensor 30 that exists in the object M (in the catheter) detects the magnetic field intensity. The data reception unit 2002 receives the information indicating the magnetic field intensity from the operation assisting apparatus magnetic sensor 30 via a catheter 31. The coordinate calculation unit 2003 calculates the coordinate positions of the operation assisting apparatus magnetic sensor 30 based on the magnetic field intensity information received by the data reception unit 2002.

The coordinate position information of the operation assisting apparatus magnetic sensor 30 thus acquired by the magnetic field generator/detector 201 is output to the image processing unit 104 provided in the radiation imaging apparatus 10. The image processing unit 104 plots the coordinate position information on a radiation image captured in advance and displays it on the monitor 105.

The switch SW 202 controls driving/stop of the magnetic field generator/detector 201 under the control of the image processing unit 104.

<Method of Deciding Timing to Restrict Driving of Operation Assisting Apparatus 20>

A signal to restrict driving of the operation assisting apparatus 20 is generated by the image processing unit 104 and output to the switch SW 202 that controls driving of the magnetic field generator/detector 201. The restriction signal output period is decided in accordance with the image noise amount of the acquired radiation image.

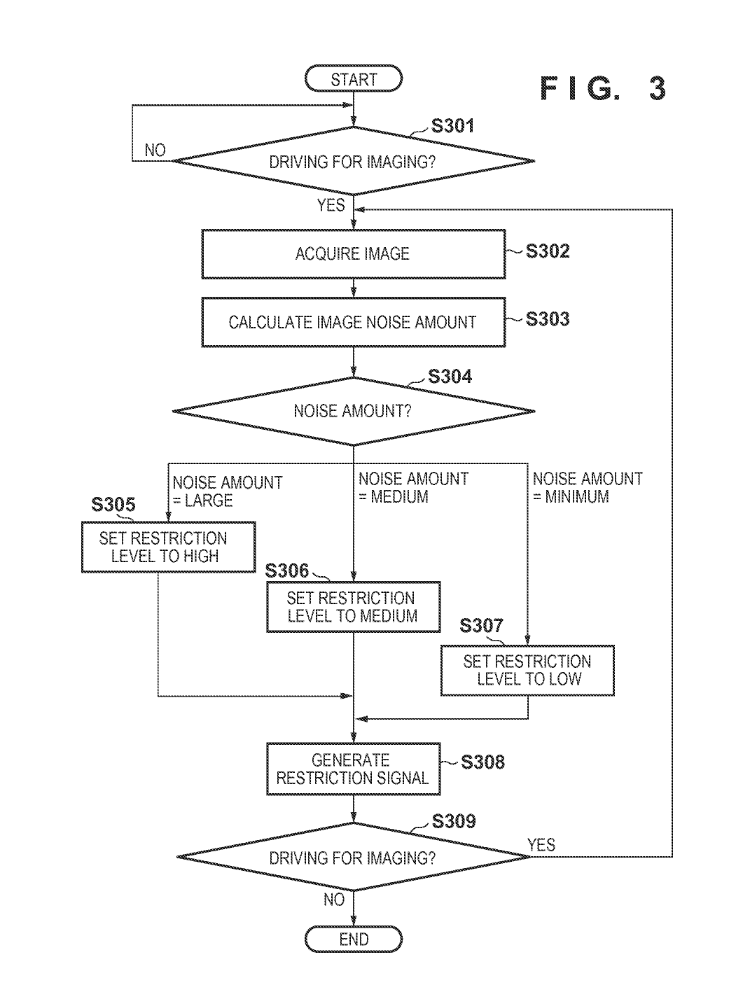

The procedure of processing executed by the radiation imaging apparatus 10 according to the first embodiment will be described below with reference to the flowchart of FIG. 3.

In step S301, the driving control unit 103 determines whether the radiation detector 102 is being driven for imaging. Upon determining that the radiation detector 102 is being driven for imaging (step S301: YES), the process advances to step S302. On the other hand, upon determining that the radiation detector 102 is not being driven for imaging (step S301: NO), the processing waits.

In step S302, the image processing unit 104 processes detection signals acquired from the radiation detector 102 and acquires a radiation image.

In step S303, the image processing unit 104 calculates the image noise amount of the acquired radiation image. As one method, the image noise amount is calculated by the standard deviation of pixel values in a region of interest (ROI).

In step S304, the image processing unit 104 determines the magnitude of the image noise amount. In this embodiment, the image noise amounts are classified into three levels, high, medium, and low. When the image noise amount is equal to or larger than a first value, it is determined that the image noise amount is large. When the image noise amount is smaller than the first value and equal to or larger than a second value, it is determined that the image noise amount is medium. When the image noise amount is smaller than the second value, it is determined that the image noise amount is small. Upon determining that the image noise amount is large, the process advances to step S305. Upon determining that the image noise amount is medium, the process advances to step S306. Upon determining that the image noise amount is small, the process advances to step S307.

In step S305, the image processing unit 104 sets the restriction level of driving of the operation assisting apparatus 20 to high. “Restriction level high” indicates a control status in which driving of the operation assisting apparatus 20 is restricted (stopped) throughout the period of imaging the radiation imaging apparatus 10.

In step S306, the image processing unit 104 sets the restriction level of driving of the operation assisting apparatus 20 to medium. “Restriction level medium” indicates a control status in which driving of the operation assisting apparatus 20 is restricted (stopped) during a partial period of imaging of the radiation imaging apparatus 10.

In step S307, the image processing unit 104 sets the restriction level of driving of the operation assisting apparatus 20 to low. “Restriction level low” indicates a control status in which driving of the operation assisting apparatus 20 is not restricted (stopped) even during imaging of the radiation imaging apparatus 10.

In the above-described way, the restriction signal output period is decided in accordance with the image noise amount calculated in step S303.

In step S308, the image processing unit 104 generates a restriction signal to restrict driving of the operation assisting apparatus 20 based on the output period decided in one of steps S305 to S307, and outputs the restriction signal to the operation assisting apparatus 20.

In step S309, the driving control unit 103 determines whether the radiation detector 102 is being driven for imaging. Upon determining that the radiation detector 102 is being driven for imaging (step S309: YES), the process returns to step S302. On the other hand, upon determining that the radiation detector 102 is not being driven for imaging (step S309: NO), the processing ends.

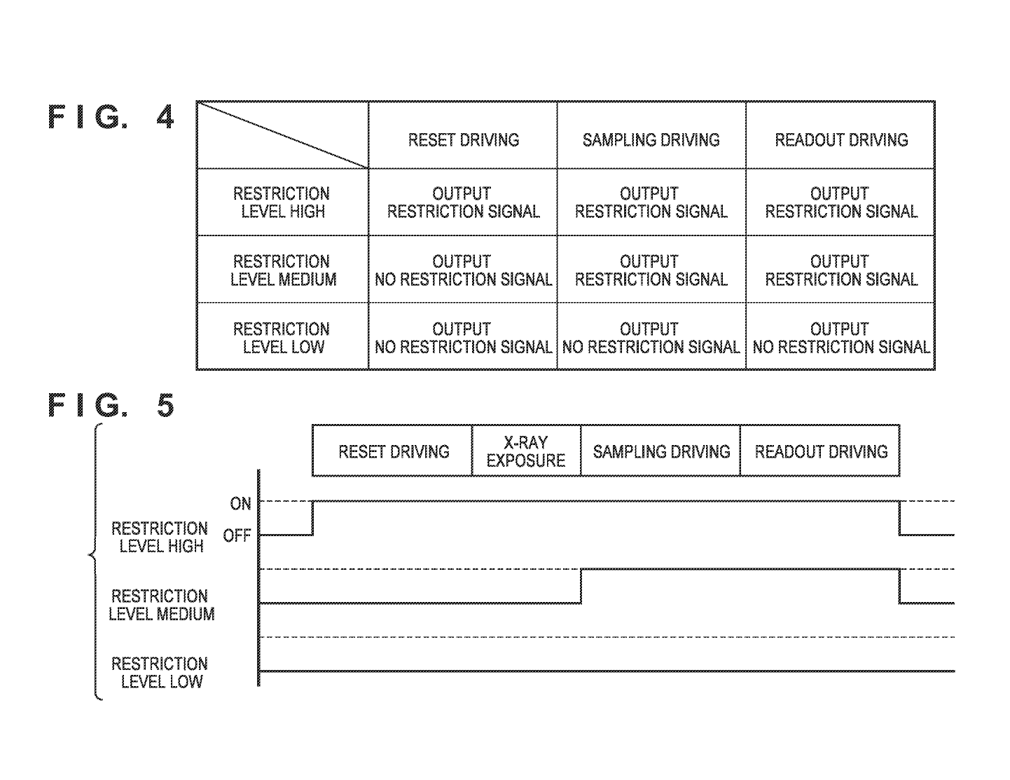

FIG. 4 is a table showing output modes that define restriction signal output timings representing whether to output the restriction signal for each driving status of the radiation imaging apparatus 10. The radiation imaging apparatus 10 can have various driving statuses such as reset driving, sampling driving, and readout driving. More specific control can be performed by outputting the restriction signal in accordance with the driving status. The information of each output mode is stored in, for example, the memory (not shown) of the driving control unit 103. The driving control unit 103 loads the information of one of the output modes shown in FIG. 4 from the memory in accordance with the restriction level decided based on the flowchart of FIG. 3, and controls the restriction signal output timing based on the loaded information. As described above, the restriction level is decided based on, for example, the noise level. Hence, the output of the electromagnetic field can appropriately be restricted in accordance with the noise level.

In the example of FIG. 4, for restriction level high, the restriction signal is output in all driving statuses such as reset driving, sampling driving, and readout driving. For restriction level medium, the restriction signal is output in sampling driving and readout driving but not in reset driving. For restriction level low, the restriction signal is output in neither of the driving statuses such as reset driving, sampling driving, and readout driving.

Especially when a magnetic field is generated in readout driving, the influence on the image quality is large. The readout driving, sampling driving, and reset driving may be ranked for restriction in this order in accordance with the time interval and time-axis relationship to the readout driving.

FIG. 5 shows an example of a restriction signal generation timing for each restriction level. The image noise amount calculated by the image processing unit 104 is used to set the restriction signal output period for the next image acquisition. At the time of image acquisition, the driving of the radiation detector 102 is roughly divided into reset driving, sampling driving, and readout driving. Three restriction levels are set in accordance with the calculated image noise amount, and the restriction signal output period is set for each driving status in accordance with the restriction level.

For example, for restriction level high, the restriction signal is generated from the reset driving period to the readout driving period. For restriction level medium, the restriction signal is not generated in the reset driving period, and the generation starts from the start of the sampling driving period. For restriction level low, the restriction signal is not generated from the reset driving period to the readout driving period.

As described above, when driving of the operation assisting apparatus is restricted based on the driving status of the radiation imaging apparatus and the restriction level corresponding to the image noise amount, the period in which the period to perform radiation imaging overlaps the period to drive the operation assisting apparatus shortens. This makes it possible to suppress noise in the radiation image even when the radiation imaging apparatus is used together with the separate operation assisting apparatus.

Note that control may further be performed to start outputting the restriction signal in accordance with the end of radiation irradiation for the radiation imaging apparatus. Control may be performed not to output the restriction signal during the period of radiation irradiation for the radiation imaging apparatus. Control may be performed to output the restriction signal during at least one of the periods of reset driving, sampling driving, and readout driving by the radiation imaging apparatus. The apparatus may further include a selection unit that selects one of a plurality of modes for determining the output timing of the restriction signal, and control may be performed to output the restriction signal in the mode according to selection by the selection unit.

### Second Embodiment

In the first embodiment, the method of deciding the restriction level based on the image noise amount has been described. In the second embodiment, an arrangement that decides the restriction level based on the detection value of a radiation detector magnetic sensor provided on a radiation detector will be explained. Note that the same reference numerals as in the first embodiment denote the same constituent elements in the second embodiment.

<Restriction Level Decision Method by Magnetic Sensors>

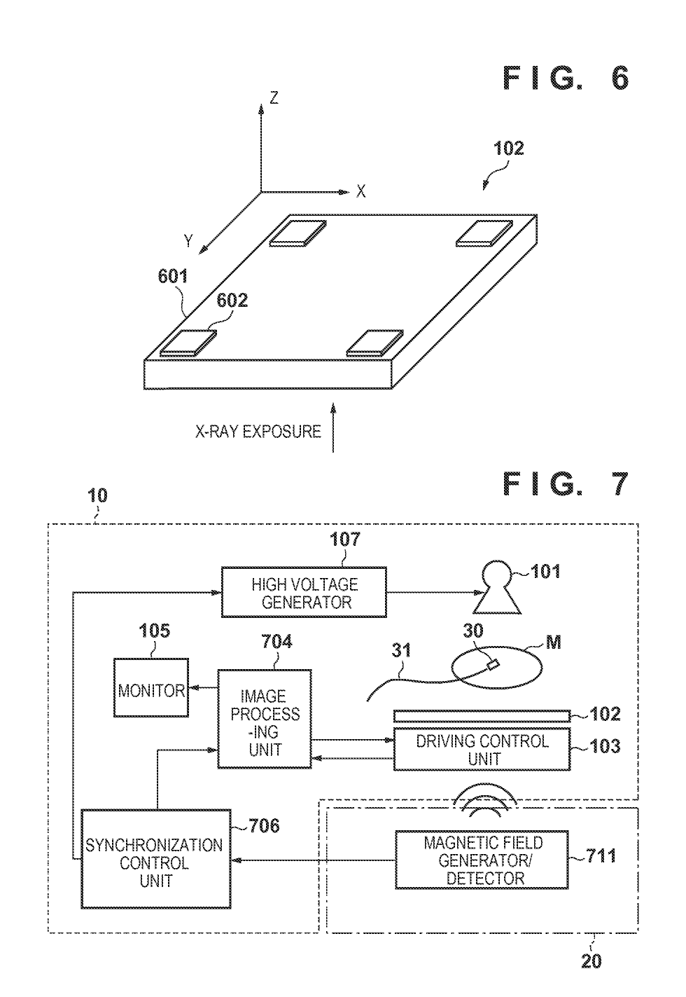

FIG. 6 shows the installation positions of radiation detector magnetic sensors. A radiation detector 102 includes a radiation detector case 601. One or a plurality of radiation detector magnetic sensors 602 are arranged on the X-Y plane located on the positive side in the Z-axis direction of the radiation detector case 601.

The radiation detector magnetic sensors 602 are arranged at the four corners of a surface that is provided on the opposite side of the surface exposed to a radiation and is readily affected by magnetic field application (in FIG. 6, the surfaces perpendicular to the Z-axis are assumed to be the surfaces readily affected by magnetic field application). The highest one of the detection values of the four radiation detector magnetic sensors 602 is confirmed, and the restriction level is decided in accordance with a predetermined threshold.

As in the first embodiment, when the highest detection value is equal to or larger than a first value, it is determined that the restriction level is high. When the highest detection value is smaller than the first value and equal to or larger than a second value, it is determined that the restriction level is medium. When the highest detection value is smaller than the second value, it is determined that the restriction level is low.

Alternatively, when only one radiation detector magnetic sensor 602 is provided, the restriction level is decided based on the detection value of the radiation detector magnetic sensor 602 in a similar manner. The method of deciding the restriction timing of an operation assisting apparatus 20 is the same as in the first embodiment.

Note that the radiation detector magnetic sensor 602 preferably has a high sensitivity characteristic to the frequency band of a magnetic field generated by a magnetic field generator/detector 201.

As described above, when driving of the operation assisting apparatus is restricted based on the detection value of the radiation detector magnetic sensor, the period in which the period to detect the radiation overlaps the period to drive the operation assisting apparatus shortens. This makes it possible to suppress noise in the radiation image even when the radiation imaging apparatus is used together with the separate operation assisting apparatus.

### Third Embodiment

In the first and second embodiments, the arrangement for outputting a signal to restrict driving from the radiation imaging apparatus side to the operation assisting apparatus side has been described. In the third embodiment, an arrangement for outputting a signal to restrict radiation generation or readout driving from the operation assisting apparatus side to the radiation imaging apparatus side will be explained. Note that the same reference numerals as in the first embodiment denote the same constituent elements in the third embodiment.

In this embodiment, the operations of an image processing unit 704 and a synchronization control unit 706 included in a radiation imaging apparatus 10 shown in FIG. 7 are different from those in the first embodiment. In addition, the operation of a magnetic field generator/detector 711 included in the radiation imaging apparatus 10 is different from that in the first embodiment.

The image processing unit 704 performs various kinds of processing such as correction for detection signals, thereby acquiring image data. The acquired image data is stored in a storage unit (not shown) provided in the image processing unit 704 and also output to a monitor 105. Unlike the first embodiment, the image processing unit 704 does not execute arithmetic processing concerning the image noise amount.

The synchronization control unit 706 is connected to a high voltage generator 107 and a radiation detector 102 directly or via the image processing unit 704, and synchronizes radiation irradiation with radiation detection. More specifically, the synchronization control unit 706 causes the high voltage generator 107 to apply a tube voltage to a radiation tube 101 in accordance with the timing of detection signal collection of the radiation detector 102. This makes it possible to indirectly give the radiation tube 101 an instruction to irradiate an object with the radiation at a predetermined irradiation time. The synchronization control unit 706 is also connected to the image processing unit 704 and synchronizes image data output and the like with the radiation irradiation and radiation detection.

The synchronization control unit 706 receives, from an operation assisting apparatus 20, a restriction signal output during driving of the magnetic field generator/detector 711. Upon receiving the restriction signal, the synchronization control unit 706 stops the irradiation instruction to the radiation tube 101. In addition, the synchronization control unit 706 instructs the image processing unit 704 to continuously display, on the monitor 105, the image already displayed immediately before the restriction signal reception.

In addition, upon receiving the display continuation instruction resulted from the reception of the restriction signal from the synchronization control unit 706, the image processing unit 704 requests a driving control unit 103 to stop image readout driving. At this time, image data read out halfway may be discarded.

The restriction signal is generated by the magnetic field generator/detector 711 during its driving period, that is, during the period is which the magnetic field is being generated. As described above, the signal to restrict radiation generation or readout driving is output from the operation assisting apparatus side to the radiation imaging apparatus side during the driving period of the magnetic field generator/detector. Since control can be performed not to make the radiation imaging apparatus perform radiation generation or readout during magnetic field generation, noise in the acquired radiation image can be reduced.

According to the present invention, it is possible to suppress noise in a radiation image even when the radiation imaging apparatus is used together with the separate operation assisting apparatus.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-246662 filed on Nov. 8, 2012, which is hereby incorporated by reference herein in its entirety.

What is claimed is:

1. A radiation imaging apparatus that captures a radiation image of an object, comprising:
an output unit configured to, in accordance with a driving status of the radiation imaging apparatus, output a signal to restrict driving of an operation assisting apparatus that generates a magnetic field when assisting an operation for the object.

2. The apparatus according to claim 1, wherein said output unit is configured to start outputting the restriction signal in accordance with an end of radiation irradiation for the radiation imaging apparatus.

3. The apparatus according to claim 1, wherein said output unit is configured not to output the restriction signal during a period of radiation irradiation for the radiation imaging apparatus.

4. The apparatus according to claim 3, wherein said output unit is configured to output the restriction signal during at least one of periods of reset driving, sampling driving, and readout driving by the radiation imaging apparatus.

5. The apparatus according to claim 1, further comprising a selection unit configured to select one of a plurality of modes for determining an output timing of the restriction signal by said output unit.

6. The apparatus according to claim 1, further comprising:
a sensor configured to output an image signal by a radiation; and
a driving control unit configured to control said sensor,
said driving control unit causing said sensor to perform electronic shutter driving.

7. The apparatus according to claim 1, further comprising:
a calculation unit configured to calculate an image noise amount of the captured radiation image; and
a setting unit configured to set a restriction level to restrict the driving of the operation assisting apparatus based on the image noise amount,
wherein said output unit is configured to output the signal to restrict the driving of the operation assisting apparatus during a period predetermined in accordance with the restriction level and the driving status of the radiation imaging apparatus.

8. The apparatus according to claim 1, further comprising:
a magnetic sensor configured to detect an intensity of the magnetic field generated by the operation assisting apparatus; and
a setting unit configured to set a restriction level based on the intensity of the magnetic field,
wherein said output unit is configured to output the signal to restrict the driving of the operation assisting apparatus during a period predetermined in accordance with the restriction level and the driving status of the radiation imaging apparatus.

9. The apparatus according to claim 1, wherein the operation assisting apparatus comprises a catheter navigation system.

10. An operation assisting apparatus that generates a magnetic field when assisting an operation for an object, comprising:
an output unit configured to, in accordance with a driving status of the operation assisting apparatus, output a signal to restrict driving of a radiation imaging apparatus that captures a radiation image of the object.

11. A control method of a radiation imaging apparatus that captures a radiation image of an object, comprising:
outputting, in accordance with a driving status of the radiation imaging apparatus, a signal to restrict driving of an operation assisting apparatus that generates a magnetic field when assisting an operation for the object.

12. A control method of an operation assisting apparatus that generates a magnetic field when assisting an operation for an object, comprising:
outputting, in accordance with a driving status of the operation assisting apparatus, a signal to restrict driving of a radiation imaging apparatus that captures a radiation image of the object.

13. A non-transitory computer-readable storage medium storing a computer program that causes a computer to execute the control method of a radiation imaging apparatus of claim 11.

14. A non-transitory computer-readable storage medium storing a computer program that causes a computer to execute the control method of an operation assisting apparatus of claim 12.
